(19) United States
(12) Patent Application Publication
(10) Pub. No.: US 2010/0238406 A1
(43) Pub. Date: Sep. 23, 2010

(54) FOCUS ASSISTING DEVICE

(75) Inventors: Chih-Kuang Chang, Tu-Cheng (TW); Li Jiang, Shenzhen City (CN); Dong-Hai Li, Shenzhen City (CN); Xiao-Guang Xue, Shenzhen City (CN); Yi-Rong Hong, Shenzhen City (CN)

Correspondence Address: Altis Law Group, Inc.;ATTN: Steven Reiss, 288 SOUTH MAYO AVENUE, CITY OF INDUSTRY, CA, 91789, US

(73) Assignees: HONG FU JIN PRECISION INDUSTRY (ShenZhen) CO.LTD., Shenzhen City (CN); HON HAI PRECISION INDUSTRY CO., LTD., Tu-Cheng (TW)

(21) Appl. No.: 12/421,581

(22) Filed: Apr. 9, 2009

(30) Foreign Application Priority Data
Mar. 19, 2009 (CN) 200910300951.X

(51) Int. Cl. A61B 3/10; G02B 5/00

(52) U.S. Cl. 351/211; 359/893

(57) ABSTRACT

A focus assisting device is configured to assist a vision measuring system to focus on an object with smooth low-contrast surfaces. The focus assisting device includes a plurality of transparent portion and a plurality of nontransparent portion.

### CROSS-REFERENCE TO RELATED APPLICATION

Relevant subject matter is disclosed in a co-pending U.S. patent application (Attorney Docket No. US23325) filed on the same date and having a title of “VISION MEASURING SYSTEM AND ASSISTANT FOCUSING SYSTEM THEREOF”, which is assigned to the same assignee as this patent application.

### BACKGROUND

1. Technical Field

Embodiments of the present disclosure relate to focusing device, and more particularly, to a focus assisting device.

2. Description of Related Art

Auto-focus systems are provided in optical measuring instruments such as what are used in vision measuring systems. A contrast method to focus lenses based on the contrast of an image of an object surface to be measured is well known in the auto-focus system field. However, it is difficult for the contrast method to focus precisely on an essentially low contrast surface such as a mirror-finish surface or a glass surface.

### BRIEF DESCRIPTION OF THE DRAWINGS

FIG. 1 is an isometric view of an exemplary embodiment of a focus assisting device.

FIG. 2 is a cross-sectional view of a vision measuring device applying the focus assisting device of FIG. 1.

### DETAILED DESCRIPTION

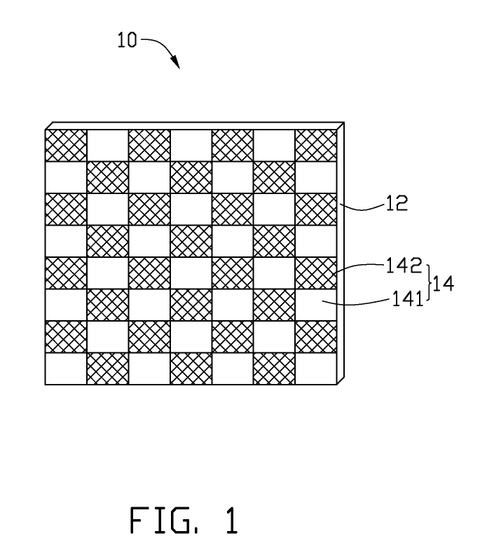

Referring to FIG. 1, an exemplary embodiment of a focus assisting device 10 includes a slide 12 which may be constructed as any typical specimen slide used in a laboratory. The focus assisting device 10 further includes a pattern 14 attached on a surface of the slide 12. The slide 12 is a transparent element, such as is normally used for a glass slide. The pattern 14 includes a plurality of square transparent portions 141 and a plurality of square nontransparent portions 142. The plurality of square transparent portions 141 and the plurality of square nontransparent 142 are about 0.10 millimeter long on each side and arranged in checkerboard fashion on the slide 12. In other embodiments, the slide 12 can be another type of transparent member having different shape and size. The pattern 14 also can be formed in some other pattern or image, and non-transparent portions of the pattern 14 can be formed by etching a surface of the slide 12. Alternatively, non-transparent material could be used to make the slide 12 and holes defined in the slide 12 to achieve a pattern of contrasting portions.

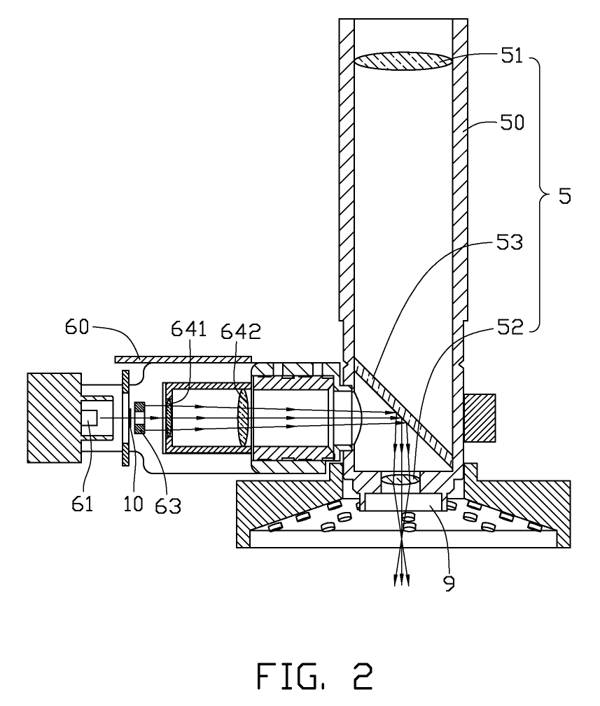

Referring to FIG. 2, the vision measuring system applying the focus assisting slide 10 to focus on an object 9 with smooth low-contrast surfaces. The vision measuring system includes an optical apparatus 5, an illuminating device, and an image capture apparatus such as are commonly used in vision measuring systems. The optical apparatus 5 includes a cylinder-shaped first shell 50, first and second object lenses 51 and 52 mounted to first and second ends of the first shell 50, and a partial reflector 53. The partial reflector 53 is located between the first object lens 51 and the second object lens 52 in the first shell 50. There is an angle of about 45° between the optical axis of the optical system 5 and the partial reflector 53.

The illuminating device is configured to position the focus assisting device 10 and project the pattern 14 of the focus assisting device 10 on the surface of the object 9. The illuminating device includes a second shell 60, a first illuminator 61, a second illuminator 63, a third object lens 641, and a fourth object lens 642. The first illuminator 61, the second illuminator 63, the third object lens 641 and the fourth object lens 642 are located in the second shell 60 in that order. The focus assisting device 10 is located between the first illuminator 61 and the second illuminator 63, to allow light projected by the first illuminator 61 to pass through the focus assisting device 10 to project the pattern 14 of the focus assisting device 10 on the object 9.

For smooth low-contrast objects such as the object 9, the first illuminator 61 and the second illuminator 63 are turned on. Light from the first illuminator 61 passes through the focus assisting slide 10, the third object lens 641, the fourth object lens 642, the semi-transparent reflector 53, and the second object lens 52 to be projected onto the surface of the object 9. As a result, the pattern 14 of the focus assisting device 10 is projected on the surface of the object 9. In addition, light from the second illuminator 63 passes through the third and fourth object lenses 641 and 642, the semi-transparent reflector 53, and the second object lens 52 to be projected onto the surface of the object 9, to illuminate the object 9. The optical system 5 focuses light reflected by the surface of the object 9 on an image capturing surface of the image capture apparatus, which captures an image of the surface of the object 9 and converts the image into electronic signals, to transmit the electronic signals to a computer system. The computer system computes a focus of the object 9 according to the contrast of the image of the object 9 using any one or more methods known in the art.

The foregoing description of the exemplary embodiments of the disclosure has been presented only for the purposes of illustration and description and is not intended to be exhaustive or to limit the disclosure to the precise forms disclosed. Many modifications and variations are possible in light of the above everything. The embodiments were chosen and described in order to explain the principles of the disclosure and their practical application so as to enable others of ordinary skill in the art to utilize the disclosure and various embodiments and with various modifications as are suited to the particular use contemplated. Alternative embodiments will become apparent to those of ordinary skills in the art to which the present disclosure pertains without departing from its spirit and scope. Accordingly, the scope of the present disclosure is defined by the appended claims rather than the foregoing description and the exemplary embodiments described therein.

What is claimed is:

1. A focus assisting device configured to assist a vision measuring system to focus on an object with smooth low-contrast surfaces, comprising:
a plurality of transparent portions and a plurality of nontransparent portions.

2. The focus assisting device of claim 1, further comprising a transparent slide, wherein the plurality of transparent and nontransparent portions are attached on the transparent slide body.

3. The focus assisting device of claim 2, wherein the transparent slide is a glass slide.

4. The focus assisting slide of claim 2, wherein the plurality of transparent portions and the plurality of nontransparent portions are arranged in checkerboard fashion on the transparent slide.

5. The focus assisting slide of claim 4, wherein the plurality of square transparent portions and the plurality of square nontransparent are about 0.10 millimeter long on each side.

6. A method of focusing on a low-contract object, comprising:
providing a focus assisting slide which comprises a plurality of transparent portions and a plurality of nontransparent portions;
projecting the slide image onto the object; and
focusing on the object by using the contrast of the slide image projected on the object.

7. The method of claim 6, wherein the plurality of transparent portions and the plurality of nontransparent portions are arranged in checkerboard fashion on a transparent slide.

8. The method of claim 6, wherein the plurality of transparent portions and the plurality of nontransparent portions are about 0.10 millimeter long on each side.
